(19) United States
(12) Patent Application Publication
(10) Pub. No.: US 2003/0139391 A1
(43) Pub. Date: Jul. 24, 2003

(54) Efficacious dosage regimen of galantamine that reduces side effects

(76) Inventors: Wim Louis Julien Parys, Gaithersburg, MD (US); Michael Pontecorvo, Lawrenceville, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/263,920

(22) Filed: Oct. 3, 2002

Related U.S. Application Data
(63) Continuation of application No. PCT/EP01/03552, filed on Mar. 28, 2001.
(60) Provisional application No. 60/194,261, filed on Apr. 3, 2000.

(51) Int. Cl. A61K 31/55

(52) U.S. Cl. 514/214.03

(57) ABSTRACT

Galantamine has be used in the treatment of a number of chronic diseases. The use of this drug is associated with side effects such as, nausea or vomiting, and headaches. It has been demonstrated that by slowly introducing the patient to the drug these side effects can be reduced. It has also been shown that this slower titration results in the ability to use a lower effective dose of the drug.

The present invention relates to a slower titration regimen that results in a safe and effective use of galantamine at from about 16 mg/day to about 24 mg/day for the treatment of galantamine responsive conditions, with improved tolerability of the drug.

### BACKGROUND OF THE INVENTION

Galantamine is a reversible cholinesterase inhibitor that can be isolated from a number of different plant sources, including daffodil bulbs. Galantamine interacts competitively with the enzyme, acetylcholinesterase, and demonstrates a 10 to 50 fold selectivity for acetyl vs. butyryl cholinesterase.

Galantamine has been used for the treatment of a number of chronic diseases, where life-long treatment may be necessary. Galantamine has been shown to be effective in the treatment of arthritic disorders (Canadian Patent application 2,251,114); fatigue syndromes (Canadian Patent application 2,108,880); mania (Canadian Patent application 2,062,094);schizophrenia (Canadian Patent application 2,108,880); memory dysfunction, including Alzheimer's Disease (U.S. Pat. No. 4,663,318); alcoholism (Canadian Patent 2,039,197); nicotine dependence (Canadian Patent application 2,153,570); disorders of attention (PCT published application WO 99/21561) and jet lag (Canadian Patent application 2,193,473).

The use of Galantamine for such treatment is complicated by the occurrence of numerous side effects which effect the patients tolerability of the drug. It is known that the side effects, such as nausea or vomiting and headaches, can be reduced if the drug is introduced at a low dose and the dosage gradually increase to the optimal active dose. However, there has been no determination of any such suitable dosage regimen, provided in earlier studies.

In Applicant's own prior studies, patients received a daily dose of 0 (placebo), 18 mg, 24 mg, and 36 mg of galantamine free base, divided over three equal doses, following a two week titration period, wherein the patient received an initial dose of 8 mg/day for the first week, followed by 16 mg/day in the second week to the final dose thereafter. Cognitive performance, as measured by the primary variable, the ADAS-cog (Rosen, W. G. et al., Amer, J. Psychiatry, 146: 1356-1364, 1984), was statistically superior in the 24 mg galantamine group vs. the placebo group. A similar magnitude of effect was seen for the 36 mg galantamine group, but a relatively high dropout rate (50%) due to cholinergic side effects was also observed at the higher dose. A dose of 18 mg/day of galantamine showed a statistical improvement over the placebo, but this effect was numerically smaller relative to that seen for the other galantamine groups. An improvement in tolerability at this dose was seen, but this dose was suboptimal from an efficacy standpoint.

Thus, it is an object of the present invention to evaluate the safety and efficacy of various doses of galantamine when a slow titration regimen is employed.

### SUMMARY OF THE INVENTION

Thus, according to the present invention there is provided a slower titration regimen that results in a safe and effective use of galantamine at from about 16 mg/day to about 24 mg/day for the treatment of galantamine responsive conditions, with improved tolerability of the drug.

In one embodiment of the present invention there is provided a daily dosage of from about 16 mg to about 24 mg of galantamine, wherein said dosage is reached after a titration of from about 2 to about 10 weeks with the initial dose at about 8 mg increasing to a final dose of from about 16 mg to about 24 mg.

In a further embodiment of the present invention there is provided a use of a daily dosage of from about 16 mg to about 24 mg of galantamine, wherein said dosage is reached after a titration of 2 to 10 weeks with the initial dose at about 8 mg increasing to a final dose of about 16 mg to about 24 mg for the treatment of for the treatment of galantamine responsive conditions.

In yet a further embodiment of the present invention there is provided a use of a daily dosage of from about 16 mg to about 24 mg of galanamine, wherein said dosage is reached after a titration of 2 to 10 weeks with the initial dose at about 8 mg increasing to a final dose of about 16 mg to about 24 mg for the treatment of Alzheimer's Disease.

### BRIEF DESCRIPTION OF THE DRAWINGS

These and other features of the invention will become more apparent from the following description in which reference is made to the appended drawings wherein:

FIG. 1 shows the mean change from baseline by treatment group over time in ADAS-cog/11 (observed case).

FIG. 2 shows the mean change from baseline by treatment group over time in CIBIC-plus (observed case).

FIG. 3 shows the cumulative percentage of patients with specified changed from baseline at Month 5 in ADAS-cog/11 scores.

FIG. 4 shows the change in ADL performance from baseline over time at Month 5.

### DESCRIPTION OF PREFERRED EMBODIMENT

The present invention relates to a slower titration regimen that results in a safe and effective use of galantamine at from about 16 mg/day to about 24 mg/day for the treatment of galantamine responsive conditions, with improved tolerability of the drug.

Galantamine, a tertiary alkaloid, has been isolated form the bulbs of the Caucasian snowdrops Galantanus woronowi (Proskurnina, N. F. and Yakoleva, A. P. 1952, Alkaloids of Galanthus woronowi. II. Isolation of a new alkaloid. (In Russian.) Zh.Obschchei Khim. (J. Gen. Chem.) 22, 1899-1902). It has also been isolated from the common snowdrop Galanthus nivalis (Boit, 1954). Galantamine is a well-known acetylcholinesterase inhibitor which is active at nicotinic receptor sites but not on muscarinic receptor sites. It is capable of passing the blood-brain barrier in humans, and presents no severe side effects in therapeutically effective dosages.

Galantamine has been used extensively as a curare reversal agent in anaesthetic practice in Eastern bloc countries (cf. review by Paskow, 1986) and also experimentally in the West (cf. Bretagne and Valetta, 1965: Wislicki, 1967; Consanitis. 1971).

Galantamine has been marketed by the company Waldheim (Sanochemia Gruppe) as Nivalin™ in Germany and Austria since the 1970s for indications such as facial neuralgia.

In the present invention when we refer to galantamine we include within this term galantamine itself, derivatives and salts thereof, such as halides, for example galantamine hydrobromide.

For the purposes of the present invention galantamine and derivates and salts thereof may be formulated according to convention methods of pharmacy, together where appropriate with one or more pharmaceutically acceptable carriers, excipients or diluents, as is known in the art. Such formulations can take the form of tablets, capsules, solutions, or lozenges, pessaries, creams, suppositories or transdermal formulations, depending on the route of administration.

Galantamine has been used for the treatment of a number of chronic diseases, where life-long treatment may be necessary. Galantamine has been shown to be effective in the treatment of arthritic disorders (Canadian Patent application 2,251,114); fatigue syndromes (Canadian Patent application 2,108,880); mania (Canadian Patent application 2,062,094); schizophrenia (Canadian Patent application 2,108,880); memory dysfunction, including Alzheimer's Disease (U.S. Pat. No. 4,663,318); alcoholism (Canadian Patent 2,039,197); nicotine dependence (Canadian Patent application 2,153,570); disorders of attention (PCT published application WO 99/21561) and jet lag (Canadian Patent application 2,193,473). In all of these prior uses the patients tolerability of the drug was noted as a limitation.

According to the present invention, the tolerability or safety of the drug can be improved if the patient is introduced to drug slowly over a number of weeks. Thus, in one aspect of the invention the patient is introduced to galantamine slowly from about 2 week to about 10 week, wherein the dose is increase over this period.

In one embodiment of the present invention the patient receives a dose of about 8 mg/day for from about 2 weeks to about 4 weeks, followed by a dose of about 16 mg/day for from about 2 weeks to about 4 weeks, followed by a maintenance dose of about 24 mg/day thereafter.

In one example of this embodiment the patient receives a dose of about 8 mg/day for about 4 weeks, followed by a dose of about 16 mg/day for about 4 weeks, followed by a maintenance dose of about 24 mg/day thereafter.

In a further embodiment of the present invention the patient receives a dose of about 8 mg/day for from about 2 weeks to about 4 weeks, followed by a maintenance dose of about 16 mg/day thereafter. In one example of this embodiment the patient receives a dose of about 8 mg/day for about 4 weeks, followed by a maintenance dose of about 16 mg/day thereafter.

It was found according to the present invention that the patients' tolerability of galantamine was improved with the slower titration schedule employed. Further it was found that a maintenance dose of about 16 mg/day was effective in the treatment of galantamine responsive conditions, where earlier studies had shown that a dose of 18 mg/day was sub optimal from an efficacy standpoint.

The present invention is illustrated by the following example, which is not to be construed as limiting.

### EXAMPLES

Patients diagnosed with Alzheimer's Disease (approximately 910) were randomized to one of four treatment arms: placebo; 8 weeks titration to galantamine 24 mg/day; 4 weeks titration to galantamine 16 mg/day, or galantamine 8 mg/day, no titration needed, for five months. Patients included in this study must have been diagnosed with Alzheimer's Disease, had an Alzheimer's Disease Assessment Scale (Rosen, W. G. et al., Amer. J. Psychiatry, 141: 1356-1364, 1984) cognitive portion (ADAS-cog-11) score of at least 18 and had a history of cognitive decline that was gradual at the onset and progressive over a period of at least six months.

The titration schedules for the various treatment arms are as follows:

Subjects in the Placebo group received 21 weeks (5 months) of placebo medication. Subjects in group Gal 24 received 4 weeks of 8 mg/day galantamine (4 mg, twice daily (bid)), 4 weeks of 16-mg/day galantamine (8 mg, bid) and 13 weeks of 24 mg/day galantamine (12 mg, bid). Subjects in group Gal 16 received 4 weeks of 8 mg/day galantamine (4 mg, bid) and 17 weeks of 16-mg/day galantamine (8 mg, bid). Subjects in group Gal 8 received 8 mg/day (4 mg, bid) immediately upon randomization and continued on that dose for 21 weeks.

All patients were monitored throughout the study, with follow-up and cognitive evaluation at four weeks, three months and five months after the start of the study.

The primary efficacy endpoints were the change from baseline ADAS-cog/11 and the CIBIC-plus score (Clinician's Interview Based Impression of Change Plus Family Input) at month five. These two tests together with the Mini-Mental State Examination (MMSE), which was performed at the screening stage, are discussed below:

The ADAS consists of two parts—a cognitive subscale and a behavioral subscale. The behavioral subscale was not be used in this study. The cognitive subscale, the ADAS—cog-11, consisted of Word Recall and Word Recognition memory tests, Object and Finger Naming, Commands, Constructional Praxis, Ideational Praxis, Orientation, Remembering Test Instructions, Spoken language Ability, Comprehension of Spoken language and Word Finding Difficulty was the primary variable in this study.

In addition to the above specified items from the ADAS-cog-11, two additional ADAS items were assessed: The Concentration and Distractibility item, originally part of the behavioral subscale, was performed and a Delayed Word Recall test (delayed recall of the word recall items) was added to give additional information regarding cognitive status. The expanded 13 item ADAS (ADAS-cog 13) was a secondary variable.

To reduce variability due to circadian fluctuations in cognitive status the ADAS was done always at the same time of the day, preferably before noon. Only a trained ADAS rater performed the test. Ideally the ADAS rater was not involved in the treatment of the subject and should have no access to AE (adverse event) reporting.

The ADAS was performed at visits 1, 2, 3, 4 and 5 (screening, baseline, week 4, week 13 and month 5 or upon early discontinuation of trial medication intake). For word recall and word recognition two parallel wordlists, list A and list B were employed. List A was used at visits 1 and 3, List B at visits 2, 4, and 5 or upon early discontinuation of trial medication intake. For practical reasons the words for word recognition was presented only once. The total score of the 11 cognitive items on the original ADAS cognitive subscale (ADAS-cog/11, Range: 0-70) was recorded.

The CIBIC-plus score was a second primary variable. An independent, experienced and properly trained clinician provided a global impression of the subject's deterioration or improvement over the course of the trial, based on separate interviews with the subject and caregivers. If helpful, the CIBIC rater audiotaped or videotaped the baseline interview for future reference.

Change from baseline was rated on an 7 point scale, where 1 indicates markedly improved, 4 indicates no change and 7 indicates markedly worse. The CIBIC-plus was performed at visit 2, 3, 4, and 5 (baseline, week 4, week 13, and month 5 or upon early discontinuation of trial medication intake). Only a trained CIBIC rater performed the test.

The MMSE is a very brief test of cognitive functions including orientation to time and place, instantaneous recall, short-term memory, and ability to perform serial subtractions or reverse spelling, constructional capacities and the use of language. The MMSE score was derived from the sum of the points assigned to each completed task. A total possible score is 30. The MMSE will be performed at visit 1 (screening).

Secondary efficacy variables include ADAS-cog/11 and the ADCS/ADL scale. The ADCS/ADL test is discussed below:

The ADCS/ADL scale is a 23-item informant-based assessment scale measuring widely applicable daily activities appropriate for patients in the mild to moderate category of Alzheimer's Disease. The 23 items were selected for measurement from the larger set of 45 items studied by Galasko et al (Alzheimer Disease and Associated Disorders, Vol 11, Suppl. 2, 1997). These individual items were scored from 0-3 to 07, depending on the question, with a possible total score of 78. A higher score indicated a higher functioning patient.

The items and scoring were as follows:

Eating (0-3)

Walking (0-3)

Toileting (0-3)

Bathing (0-3)

Grooming (0-3)

Dressing

selection of clothes (0-3)

physical performance (0-4)

Telephone (0-5)

Television (0-3)

Conversation (0-3)

Dishes (0-3)

Managing personal belongings (0-3)

Obtaining beverages (0-3)

Making a meal or snack (0-4)

Disposal of garbage (0-3)

Travel outside home (0-4)

Shopping (0-4)

Keeping appointments (0-3)

Ability to be left alone (0-3)

Current events (0-3)

Reading (0-2)

Writing (0-3)

Hobbies (0-3)

Household appliances (0-4)

All data was compared among the treatment groups—placebo, galantamine 8 mg/day, 16 mg/day and 24 mg/day.

Between treatment groups comparisons (with particular focus on differences from placebo) were done at each scheduled time interval and for each endpoint imputation scheme. These comparisons will be based on the change from baseline scores for efficacy parameters with baseline (e.g., ADAS-cog/11) and the original scored for efficacy parameters without baseline e.g., CIBIC-plus).

For continuous data, a two-way analysis of variance (ANOVA) model with treatment and investigator as factors were used to compare the treatment groups for the change from baseline data. The interaction between treatment and investigator was examined. The impact of the baseline score on change from baseline was evaluated. If the baseline score was found to be a relevant predictor (p<10), an analysis of covariance model (ANCOVA) was used to assess the treatment effects and the interaction between treatment and baseline score was examined. If the parametric methods were deemed inappropriate (normality assumption violated), nonparametric methods such as two-way ANOVA on ranked data, Van Elteren test, controlling for investigator, was used. Following ANOVA, Fisher's LSD procedure was used for pairwise comparisons between each galanthamine group and the placebo group. A linear contrast on the main effect for treatment was used to test the dose response relationship.

For ordinal categorical variables such as the CIBIC-plus score, the Van Elteren test controlling for investigator was used for the between group comparison. For the nominal data (e.g., events rates), the Cochran-Mantel-Haenszel test for general association controlling for investigator was used. A linear contrast on the proportion of patients that stay the same or improve was used to test for increasing response with increasing dose.

If a significant proportion of subjects discontinue prematurely, additional analyses were preformed to evaluate the impact on the results. In addition to the by-visit analysis, method for analysing continuous repeated measures were used to evaluate the treatment effect over time.

The safety of the drug was also monitored throughout the study. Blood samples for biochemistry and haematology and random urine sample for urinalysis were taken at each visit and at completion for all efficacy testing. Systolic and diastolic blood pressure were measured in the sitting position, pulse and vital signs were recorded at each visit.

Patient Demographics and baseline characteristics were to be well balanced across all treatment groups (Table 1). The baseline cognitive performance for these Alzheimer's disease patients was mild to moderate as measured by the MMSE and ADAS-cog/11 scores of approximately 18 and 28 to 20.

TABLE 1
Demographics and baseline characteristics

| Trial disposition and | | | | | | | | |
|---|---|---|---|---|---|---|---|---|
| patient characteristics | Placebo | | GAL 8 mg | | GAL 16 mg | | GAL 24 mg | |
| Total Number of | 286 | | 140 | | 279 | | 273 | |
| Patients | | | | | | | | |
| Completed: N (%) | 240 | (84%) | 108 | (77%) | 219 | (79%) | 212 | (78%) |
| Gender | | | | | | | | |
| Male | 108 | (38%) | 50 | (36%) | 105 | (38%) | 90 | (33%) |
| Female | 178 | (62%) | 90 | (64%) | 174 | (62%) | 183 | (67%) |
| Age: (Years) | | | | | | | | |
| Mean (SE) | 77.1 | (0.46) | 76.0 | (0.61) | 76.3 | (0.49) | 77.7 | (0.43) |
| Median (Min-Max) | 78 | (53-100) | 77 | (52-91) | 77 | (51-94) | 78 | (57-95) |
| Race | | | | | | | | |
| Black | 13 | | 5 | | 12 | | 14 | |
| Caucasian | 267 | (93%) | 132 | (94%) | 260 | (93%) | 249 | (91%) |
| Hispanic | 3 | | 3 | | 5 | | 4 | |
| Oriental | 3 | | 0 | | 1 | | 3 | |
| Other | 0 | | 0 | | 1 | | 3 | |
| Sum of MMSE: | | | | | | | | |
| Mean (SE) | 17.7 | (0.21) | 18.0 | (0.30) | 17.8 | (0.21) | 17.7 | (0.23) |
| Median (Min-Max) | 19 | (10-22) | 19.0 | (10-22) | 19 | (10-22) | 19.0 | (10-22) |
| Baseline ADAS- | | | | | | | | |
| cog/11 | | | | | | | | |
| Mean (SE) | 29.4 | (0.63) | 27.8 | (0.94) | 29.4 | (0.66) | 29.0 | (0.67) |
| Median (Min-Max) | 27 | (10-61) | 26 | (11-62) | 28 | (10-62) | 27 | (10-54) |

The number of patients randomized among the four treatment groups was 978. The total number of patients completing this trail was high (approximately 80%) with a relatively even rate of discontinuation due to adverse events was relatively infrequent and evenly distributed among all treatment groups (see Table 2).

TABLE 2
Discontinuation of trial medication

| Trial | | GAL | GAL | GAL |
|---|---|---|---|---|
| termination reasons | Placebo | 8 mg/day | 16 mg/day | 24 mg/day |
| Total patients | 286 | 140 | 279 | 273 |
| Total completed: | 240 (84%) | 108 (77%) | 219 (79%) | 212 (78%) |
| N (%) | | | | |
| Total discontinued | 46 (16%) | 32 (23%) | 60 (22%) | 61 (22%) |
| (DC): N (%) | | | | |
| DC due to | 20 (7%) | 9 (6%) | 19 (7%) | 27 (10%) |
| adverse event | | | | |
| DC due | 0 | 1 (1%) | 0 | 2 (1%) |
| to inefficacy | | | | |
| DC due to other^{a} | 23 (8%) | 18 (13%) | 29 (10%) | 20 (7%) |
| DC due to ineligible to | 0 | 0 | 4 (1%) | 2 (1%) |
| continue | | | | |
| DC due to | 3 (1%) | 4 (3%) | 7 (3%) | 10 (4%) |
| non-compliance | | | | |
| DC due to | 0 | 0 | 1 (0.4%) | 0 |
| withdrawal of | | | | |
| consent | | | | |

^{a}The majority of discontinuations due to other reasons were for withdrawal of consent.

In this study there were two primary efficacy endpoints according to widely used international standards: change in ADAS-cog/11 score at Month 5 compared to baseline and CIBIC-plus score at Month 5.

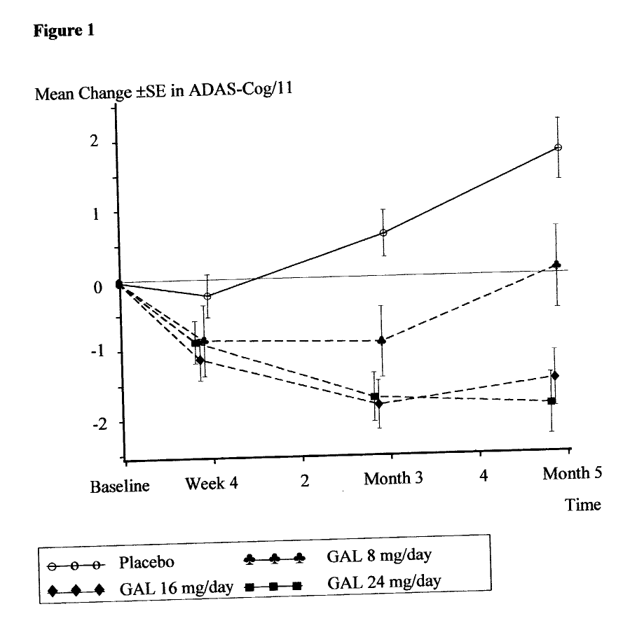

As shown in Table 3 and FIG. 1, a statistically significant treatment effect was shown for the 16 and 24 mg/day galantamine treatment groups compared with placebo for the ADAS-cog/11. Results from analysis based on the last observation carried-forward (LOCF) data corroborate the result based on observed data. The 8 mg/day galantamine group was significantly different from placebo for the observed case but not for the LOCF. Galantamine at a dose of 24 mg/day did not appear to be significantly more effective than 16 mg/day. However, the duration of exposure to the target dose differed by 1 month between the two treatment groups (two months versus three months respectively).

TABLE 3
Change from baseline in ADAS-cog/11 at Month 5

| | Placebo | GAL 8 mg | GAL 16 mg | GAL 24 mg |
|---|---|---|---|---|
| Month 5: (observed case) | n = 225 | n = 101 | n = 208 | n = 211 |
| Mean (SE) | 1.8 (0.43) | 0.1 (0.58)* | −1.5 (0.40)***^{†} | −1.8 (0.44)***^{†} |
| Month 5: (LOCF) | n = 255 | n = 126 | n = 253 | n = 253 |
| Mean (SE) | 1.7 (0.39) | 0.4 (0.52)▪ | −1.4 (0.35)***^{♭} | −1.4 (0.39)***^{♭} |

Lower score indicates better condition. P-Values based on two-way ANOVA model.
Significantly more effective than placebo: *p ≦ 0.05; **p ≦ 0.01; ***p ≦ 0.001;
Approached significance: ▪0.05 < p-value < 0. 10.
Significantly more effective than 8 mg/day: ^{†}p ≦ 0.05; ^{♭}p ≦ 0.01.

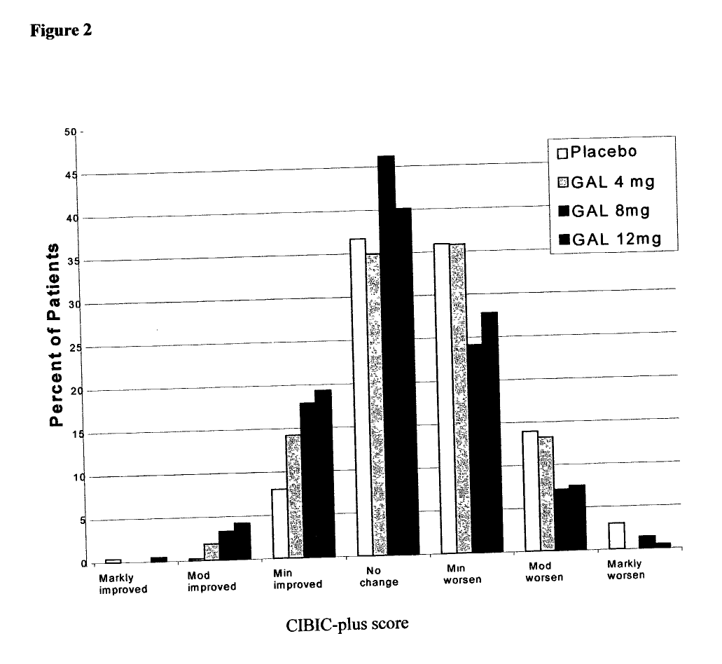

For the CIBIC-plus assessment at Month 5, the percent of patients with improved or unchanged scores was significantly greater with galantamine treatment with 16 or 24 mg/day compared with placebo or 8 mg/day of galantamine (Table 4). After 5 months of treatment, 64% to 68% of patients with 24 or 16 mg/day of galantamine showed improvement or were unchanged from baseline compared with 47% to 51% with placebo or 8 mg/day of galantamine. The analysis of imputed data at LOCF endpoint gave similar results. There was an apparent dose-related increase in the percentage of patients showing improvement or no change in the CIBIC-plus (FIG. 2).

TABLE 4
CIBIC-plus at Month 5 for improved or unchanged scores

| | Placebo | GAL 8 mg | GAL 16 mg | GAL 24 mg |
|---|---|---|---|---|
| Month 5: | n = 237 | n = 106 | n = 212 | n = 212 |
| (observed | | | | |
| case) | | | | |
| Improved/ | 112 (47%) | 54 (51%) | 143 (68%)***^{†} | 136 (64%)***^{♭} |
| no change | | | | |
| n (%) | | | | |
| Month 5: | n = 263 | n = 128 | n = 255 | n = 253 |
| (LOCF) | | | | |
| Improved/ | 128 (49%) | 68 (53%) | 169 (66%)***^{†} | 162 (64%)***^{♭} |
| no change | | | | |
| n (%) | | | | |

P-value from Van Elteren test on the 7-point scale
Significantly more effective than placebo: *p ≦ 0.05; **p ≦ 0.01; ***p ≦ 0.001.
Significantly more effective than 8 mg/day: ^{†}p ≦ 0.05; ^{♭}p ≦ 0.01.

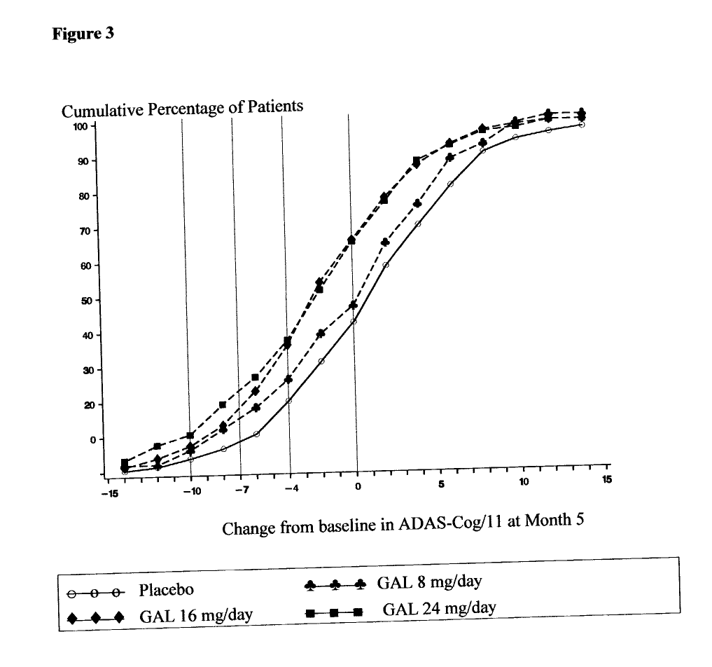

At Month 5 there were significantly more patients who responded with no change or improved scores with 16 and 24 mg/day of galantamine compared with placebo or 8 mg/day of galantamine. Patients responding with ADAS-cog/11 changes from baseline of 7 or more points occurred in 15.9% and 22.3% of patients in the 16 and 24 mg/day groups, respectively, compared with the placebo group (7.6%). There was overall a higher cumulative percentage of patients with galantamine treatment who responded with a minimum improvement of any magnitude compared with placebo (Table 5 and FIG. 3).

TABLE 5
Responders analysis based on change in ADAS-cog/11 score
from baseline at Month 5

| Definition of responder | Placebo | GAL 8 mg | GAL 16 mg | GAL 24 mg |
|---|---|---|---|---|
| | n = 225 | n = 101 | n = 208 | n = 211 |
| Change ≦ 0 points n (%) | 94 (41.8) | 47 (46.5) | 136 (65.4)***^{‡} | 137 (64.9)***^{‡} |
| Change ≦ −4 | 44 (19.6) | 26 (25.7) | 74 (35.6)***□ | 78 (37.0)***^{†} |
| points n (%) | | | | |
| Change ≦ −7 points n (%) | 17 (7.6) | 14 (13.9) | 33 (15.9)** | 47 (22.3)***□▪ |
| Change ≦ −10 points n (%) | 8 (3.6) | 6 (5.9) | 15 (7.2) | 22 (10.4)** |

P-value based on CMH test
Significantly higher percentage of responders than placebo: *p ≦ 0.05; **p ≦ 0.01; ***p ≦ 0.001;
Significantly higher percentage of responders than 8 mg/day: ^{†}p ≦ 0.05; ^{‡}p ≦ 0.01;
Approached significance: □0.05 < p-value < 0.10;
The difference between 16 and 24 mg/day approached significance ▪0.05 < p-vlaue < 0.10.

An additional secondary indication captures overall changes in Activities of daily Living (ADL) performances as measured by the Alzheimer's Disease Cooperative Study Activities of Daily Living (ACDS/ADL) scale. As mentioned above this scale is comprised of 23 items that have been tested and validated in patients with mild to moderately severe Alzheimer's disease.

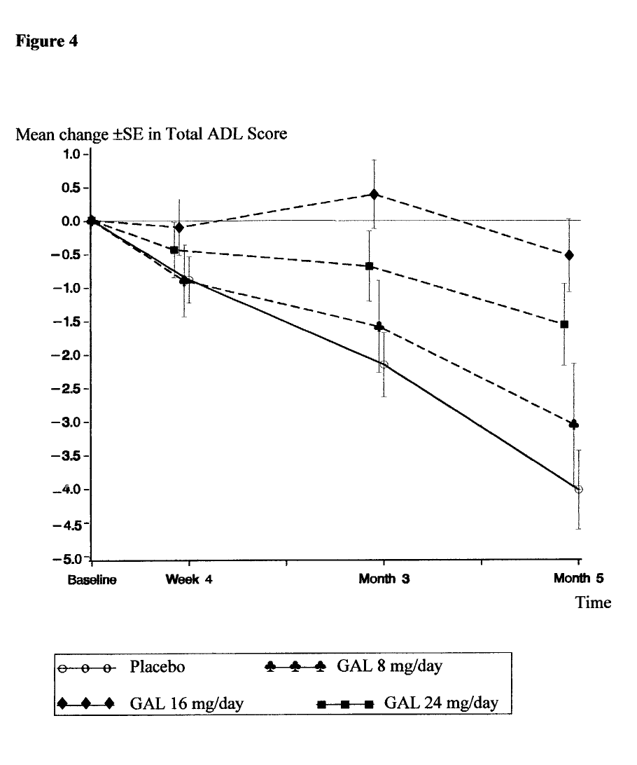

Galantamine treatment with 16 or 24 mg/day for 5 months was statistically more effective in maintaining the ADL total score at baseline levels than treatment with placebo or 8 mg/day of galantamine (Table 6). The dose-related effect of galantamine treatment is apparent in FIG. 4 that shows change of total ADL score over time.

TABLE 6
Change in Total ADL score from baseline at Month 5

| | Placebo | GAL 8 mg | GAL 16 mg | GAL 24 mg |
|---|---|---|---|---|
| Month 5: (observed case) | n = 235 | n = 106 | n = 212 | n = 212 |
| Mean (SE) | −4.0 (0.59) | −3.1 (0.91) | −0.5 (0.55)***^{†} | −1.6 (0.61)** |
| Month 5: (LOCF) | n = 262 | n = 129 | n = 255 | n = 253 |
| Mean (SE) | −3.8 (0.55) | −3.2 (0.79) | −0.7 (0.050)***^{‡} | −1.5 (0.56)** |

Higher score indicates better condition. P-Values based on two-way ANOVA model.
Significantly more effective than placebo: **p ≦ 0.01; ***p ≦ 0.001
Significantly more effective than 8 mg/day: ^{†}p ≦ 0.05; ^{‡}p ≦ 0.01.

In contrast, when a quicker titration period was used, a dose of 18 mg/day was found to be sub-optimal from an efficacy point of view.

In one such study, 285 patients were randomized into four groups: placebo, 6 mg galantamine three times daily (18 mg/day); 8 mg galantamine three times daily (24 mg/day) and 12 mg galantamine three times daily (36 mg/day). Statistical analysis of the results of the ADAS-cog score is shown below in Table 7. Statistical significance (p=0.01, triangular test) was achieved for the 24 mg/day dose, over placebo. This indicated a significant improvement of the patients' cognitive function at this dose level. The treatment with 36 mg/day in this study was terminated at an early point and thus there was insufficient information for a well defined comparison with the placebo. The treatment with 18 mg/day was not terminated early and thus these results were valid. The lower dose of 18 mg was not statistically different from the placebo.

TABLE 7
Summary Statistics of ADAS-cog

| | 18 mg/day | 24 mg/day | 36 mg/day |
|---|---|---|---|
| | (n = 88) | (n = 56) | (n = 54) |
| Z¹ | 9.9 | 13.4 | 7.8 |
| V² | 41.9 | 27.5 | 27.1 |
| Mean estimate | −1.69 | −3.34 | −1.93 |
| GAL-Placebo | (−0.04; −3.86) | (−0.78; −5.90) | (−0.60; −4.46) |
| Difference (95% | | | |
| C.I.) | | | |
| Overall SD | 6.66 | 6.83 | 6.70 |
| Actual Drug Mean | 0.5 (0.70) | 0.7 (0.9) | 1.1 (0.9) |
| (SE)³ | | | |
| Actual Placebo | 1.8 (0.7) | 2.7 (0.9) | 2.7 (0.9) |
| Mean (SE)³ | | | |
| p-value⁴ | 0.11 | 0.01 | 0.13 |

¹Z = measure of advantage of galantamine over placebo
²V = amount of information available
³Actual means are ‘raw’ means, whereas estimates of treatment difference have been adjusted for centre and interim analysis. Thus, subtracting raw means does not result in the estimated difference.
⁴Final p-value after 4th interim analysis. Treatment groups terminated early compared with appropriate placebo cohort, thus allowing for differences in the underlying patients' condition between cohorts.

Thus, the slower titration method, as disclosed in the present invention, resulted in the ability to use a lower maintenance dose, that had previously been shown to be ineffective.

The most common adverse events were evenly distributed across treatment groups with the exception of events that are associated with cholinomimetic agents. Of these related events, nausea, vomiting and anorexia showed a mild dose-related occurrence at a relatively low incidence (Table 8).

TABLE 8
Incidence of most frequent (≧ 5%) adverse
events: number (%) of patients

| Adverse event | | | | | | | | |
|---|---|---|---|---|---|---|---|---|
| (Preferred term) | Placebo | | GAL 8 mg | | GAL 16 mg | | GAL 24 mg | |
| Total all patients | 286 | | 140 | | 279 | | 273 | |
| Nausea | 13 | (4.5%) | 8 | (5.7%) | 37 | (13.3%) | 45 | (16.5%) |
| Vomiting | 4 | (1.4%) | 5 | (3.6%) | 17 | (6.1%) | 27 | (9.9%) |
| Anorexia | 9 | (3.1%) | 8 | (5.7%) | 18 | (6.5%) | 24 | (8.8%) |
| Agitation | 27 | (9.4%) | 21 | (15.0%) | 28 | (10.0%) | 22 | (8.1%) |
| Depression | 15 | (5.2%) | 4 | (2.9%) | 24 | (8.6%) | 22 | (8.%) |
| Urinary tract | 19 | (6.6%) | 11 | (7.9%) | 23 | (8.2%) | 22 | (8.1%) |
| infection | | | | | | | | |
| Dizziness | 10 | (3.5%) | 7 | (5.0%) | 15 | (5.4%) | 19 | (7.0%) |
| Injury | 12 | (4.2%) | 5 | (3.6%) | 12 | (4.3%) | 16 | (5.9%) |
| Diarrhea | 17 | (5.9%) | 7 | (5.0%) | 34 | (12.2%) | 15 | (5.5%) |
| Dyspepsia | 7 | (2.4%) | 4 | (2.9%) | 13 | (4.7%) | 15 | (5.5%) |
| Headache | 13 | (4.5%) | 5 | (3.6%) | 19 | (6.8%) | 13 | (4.8%) |
| Weight decrease | 4 | (1.4%) | 2 | (1.4%) | 15 | (5.4%) | 13 | (4.8%) |
| Fall | 14 | (4.9%) | 11 | (7.9%) | 14 | (5.05%) | 12 | (4.4%) |
| Rhinitis | 6 | (2.1%) | 9 | (6.4%) | 9 | (3.2%) | 11 | (4.0%) |
| Edema peripheral | 7 | (2.4%) | 9 | (6.4%) | 8 | (2.9%) | 7 | (2.6%) |

For most adverse events of clinical interest, as shown in Table 9, there were either no differences or slight dose-related differences between treatment groups. For bradycardia, there was a higher incidence for patients treated with galantamine compared with placebo but there was no dose-related association apparent. For syncope, there was a slight dose-related increase in incidence with 24 mg/day of galantamine, however 3 of these cases occurred at a lower dose during titration, and are therefore attributable to a lower galantamine dose. Consequently, the incidences shown in Table 6 are very likely to be over-estimates for the occurrence of syncope at the higher doses. Furthermore, 10 of 18 patients who experienced a syncopal episode were taking concomitant cardiovascular medication including bata-blockers, calcium channel antagonists, ACE inhibitors, and/or diuretics. Of these 18 patients, 11 had active cardiovascular disease listed in their past medical history. Therefore, a majority of patients who experienced syncope had either a cardiovascular condition or were taking cardiovascular medication.

TABLE 9
Adverse events of clinical interest

| Adverse event | | | | | | | | |
|---|---|---|---|---|---|---|---|---|
| (Preferred term) | Placebo | | GAL 8 mg | | GAL 16 mg | | GAL 24 mg | |
| Total all patients | 286 | | 140 | | 279 | | 273 | |
| Bradycardia | 1 | (0.3%) | 5 | (3.6%) | 7 | (2.5%) | 8 | (2.9%) |
| Convulsions | 2 | (0.7%) | 0 | | 0 | | 1 | (0.4%) |
| Fatigue | 6 | (2.1%) | 3 | (2.1%) | 10 | (3.6%) | 13 | (4.8%) |
| Muscle weakness | 3 | (1.0%) | 1 | (0.7%) | 3 | (1.1%) | 1 | (0.4%) |
| Syncope | 2 | (0.7%) | 2 | (1.4%) | 5 | (1.8%) | 9 | (3.3%) |

The incidence of serious adverse events was comparable across all treatment groups and (with the exception of syncope) showed no dose-related trends. The four most frequent serious adverse events with galantamine and with an incidence of at least 1% of patients in any group were injury, syncope, fall, and myocardial infarction (Table 10). There were no dose-related increases in GI-related serious adverse events. The only serious adverse event that showed a dose-relationship was syncope, however, for reasons already provided, these rates may be an over-estimate.

TABLE 10
Serious adverse events
(with ≧2 patients in any group)

| Adverse Event | | | | | | | | |
|---|---|---|---|---|---|---|---|---|
| (Preferred | | | | | | | | |
| term) | Placebo | | GAL 8 mg | | GAL 16 mg | | GAL 24 mg | |
| Total all patients | 286 | | 140 | | 279 | | 273 | |
| Total patients | 31 | (10.8%) | 14 | (10.0%) | 28 | (10.0%) | 35 | (12.8%) |
| with any SAE | | | | | | | | |
| Injury | 4 | (1.4%) | 1 | (0.7%) | 1 | (0.4%) | 5 | (1.8%) |
| Syncope | 2 | (0.7%) | 1 | (0.7%) | 4 | (1.4%) | 5 | (1.8%) |
| Asthenia | 1 | (0.3%) | 0 | (0.0%) | 2 | (0.7%) | 1 | (0.4%) |
| Dyspnea | 1 | (0.3%) | 0 | (0.0%) | 2 | (0.7%) | 3. | (1.1%) |
| Pneumonia | 4 | (1.4%) | 1 | (0.7%) | 2 | (0.7%) | 3 | (1.1%) |
| Gi haemorrhage | 0 | (0.0%) | 0 | (0.0%) | 0 | (0.0%) | 2 | (0.7%) |
| Vomiting | 1 | (0.3%) | 0 | (0.0%) | 2 | (0.7%) | 1 | (0.4%) |
| Abdominal pain | 1 | (0.3%) | 0 | (0.0%) | 2 | (0.7%) | 0 | (0.0%) |
| Diarrhea | 1 | (0.3%) | 0 | (0.0%) | 3 | (1.1%) | 0 | (0.0%) |
| Nausea | 1 | (0.3%) | 0 | (0.0%) | 2 | (0.7%) | 0 | (0.0%) |
| Basal cell | 0 | (0.0%) | 0 | (0.0%) | 0 | (0.0%) | 2 | (0.7%) |
| carcinoma | | | | | | | | |
| Breast | 2 | (0.7%) | 0 | (0.0%) | 0 | (0.0%) | 0 | (0.0%) |
| neoplasm | | | | | | | | |
| female | | | | | | | | |
| Fall | 3 | (1.0%) | 4 | (2.9%) | 1 | (0.4%) | 3 | (1.1%) |
| Surgical | 1 | (0.3%) | 0 | (0.0%) | 3 | (1.1%) | 0 | (0.0%) |
| intervention | | | | | | | | |
| Thrombophlebitis | 1 | (0.3%) | 0 | (0.0%) | 0 | (0.0%) | 2 | (0.7%) |
| deep | | | | | | | | |
| Transient | 1 | (0.3%) | 1 | (0.7%) | 2 | (0.7%) | 0 | (0.0%) |
| ischemic | | | | | | | | |
| attack | | | | | | | | |
| Myocardial | 2 | (0.7%) | 3 | (2.1%) | 1 | (0.4%) | 1 | (0.4%) |
| infarction | | | | | | | | |
| Agitation | 1 | (0.3%) | 2 | (1.4%) | 1 | (0.4%) | 0 | (0.0%) |
| Urinary tracq | 0 | (0.0%) | 1 | (0.7%) | 2 | (0.7%) | 0 | (0.0%) |
| infection | | | | | | | | |
| Cardiac failure | 2 | (0.7%) | 0 | (0.0%) | 3 | (1.1%) | 0 | (0.0%) |
| Dehydration | 0 | (0.0%) | 0 | (0.0%) | 3 | (1.1%) | 0 | (0.0%) |
| Sepsis | 2 | (0.7%) | 0 | (0.0%) | 0 | (0.0%) | 0 | (0.0%) |

Throughout the trial there were 11 deaths. There was no apparent dose-related patterns in the occurrences of deaths. No death was considered by the investigator to be related to trail medication.

In contrast with earlier studies with a shorter titration of the drug the incidence of side effects, particularly gastro-intestinal system disorders, were increased. A example of this is shown below in Table 11.

TABLE 11
Gastro-intestinal System Disorders

| | Placebo | 18 mg | 24 mg | 32 mg | 36 mg | flexable |
|---|---|---|---|---|---|---|
| Total | 919 | 88 | 488 | 704 | 54 | 261 |
| number | | | | | | |
| Adverse | 207 (22.5%) | 28 (31.8%) | 239 (49.0%) | 393 (55.8%) | 27 (50.0%) | 133 (51.0%) |
| events | | | | | | |
| Nausea | 92 (10.0%) | 15 (17.0%) | 172 (35.2%) | 265 (37.6%) | 20 (37.0%) | 84 (32.2%) |
| Vomiting | 37 (4.0%) | 15 (17.0%) | 93 (19.1%) | 131 (18.6%) | 9 (16.7%) | 38 (14.6%) |
| Diarrhea | 58 (6.3%) | 2 (2.3%) | 47 (9.6%) | 91 (12.9%) | 2 (3.7% | 38 (14.6%) |
| Abdominal | 29 (3.2%) | 2 (2.3%) | 36 (7.4%) | 59 (8.4%) | 1 (1.9%) | 18 (1.9%) |
| Pain | | | | | | |
| Dyspepsia | 16 (1.7%) | 5 (5.7%) | 21 (4.3%) | 32 (4.5%) | 0 (0%) | 9 (3.4%) |

The results of this example confirmed that treatment with either 16 mg/day or 24/mg day of galantamine leads to statistically significant improvements in both primary efficacy endpoints (ADAS-cog/1 I and CIBIC-plus) compared to placebo or 8 mg/day galantamine at Month 5. For the 8 mg/day galantamine group, a trend toward improvement compared to placebo was observed. The secondary endpoints were consistent with the primary results. For example, ADL results were positive for change from baseline at Month 5 for the 16 and 24 mg/day group compared with placebo.

The use of the 16 mg/day dose, after introduction with the slower dose titration schedule provides a safe and efficacious treatment, which was not known from previous studies.

There was no apparent dose-response relationship between the percentage of patients who discontinued treatment for any reason and the dose of galantamine administered. In contrast, an apparent dose-response relationship was discerned for patients with common gastrointestinal adverse events such as nausea, vomiting and anorexia.

Thus, the slower dose titration schedule uses in this study was associated with a lower rate of cholinergicaly mediated adverse events, especially those involving the gastrointestinal system. These rates are lower than those reported in previous placebo-controlled, double-blind studies.

All scientific publications and patent documents are incorporated herein by reference.

The present invention has been described with regard to preferred embodiments. However, it will be obvious to persons skilled in the art that a number of variations and modifications can be made without departing from the scope of the invention as described in the following claims.

1. A daily dosage of from about 16 mg to about 24 mg of galantamine, wherein said dosage is reached after a titration of from about 2 to about 10 weeks with the initial dose at about 8 mg increasing to a final dose of from about 16 mg to about 24 mg.

2. The daily dosage of claim 1 wherein said daily dosage is about 24 mg/day, wherein said dosage is reached after a titration of a dose of about 8 mg/day for from about 2 weeks to about 4 weeks, followed by a dose of about 16 mg/day for from about 2 weeks to about 4 weeks, followed by the daily dosage of about 24 mg/day thereafter.

3. The daily dosage of claim 2 wherein said daily dosage is about 24 mg/day, wherein said dosage is reached after a titration of a dose of about 8 mg/day for about 4 weeks, followed by a dose of about 16 mg/day for about 4 weeks, followed by the daily dosage of about 24 mg/day thereafter.

4. The daily dosage of claim 1 wherein said daily dosage is about 16 mg/day, wherein said dosage is reached after a titration of a dose of about 8 mg/day for from about 2 weeks to about 4 weeks, followed by the daily dosage of about 16 mg/day thereafter.

5. The daily dosage of claim 4 wherein said daily dosage is about 16 mg/day, wherein said dosage is reached after a titration of a dose of about 8 mg/day for about 4 weeks, followed by the daily dosage of about 16 mg/day thereafter.

6. A regimen for treating a galantamine responsive condition which comprises administering to a patient in need thereof a daily dosage of from about 16 mg to about 24 mg of galantamine, wherein said dosage is reached after a titration of from about 2 to about 10 weeks with the initial dose at about 8 mg increasing to a final dose of about 16 mg to about 24 mg.

7. The regimen of claim 6 wherein said daily dosage is about 24 mg/day, wherein said dosage is reached after a titration of a dose of about 8 mg/day for from about 2 weeks to about 4 weeks, followed by a dose of about 16 mg/day for from about 2 weeks to about 4 weeks, followed by the daily dosage of about 24 mg/day thereafter.

8. The regimen of claim 7 wherein said daily dosage is about 24 mg/day, wherein said dosage is reached after a titration of a dose of about 8 mg/day for about 4 weeks, followed by a dose of about 16 mg/day for about 4 weeks, followed by the daily dosage of about 24 mg/day thereafter.

9. The regimen of claim 6 wherein said daily dosage is about 16 mg/day, wherein said dosage is reached after a titration of a dose of about 8 mg/day for from about 2 weeks to about 4 weeks, followed by the daily dosage of about 16 mg/day thereafter.

10. The regimen of claim 9 wherein said daily dosage is about 16 mg/day, wherein said dosage is reached after a titration of a dose of about 8 mg/day for about 4 weeks, followed by the daily dosage of about 16 mg/day thereafter.

11. The regimen of claim 6 wherein the galantamine responsive condition is selected from the group consisting of arthritic disorders; fatigue syndromes; mania; schizophrenia; memory dysfunction; alcoholism; nicotine dependence; and jet lag.

12. The regimen of claim II wherein the galantamine responsive condition is Alzheimer's Disease.

13. A regimen for treating Alzheimer's Disease which comprises administering to a patient in need thereof a daily dosage of from about 16 mg to about 24 mg of galantamine, wherein said dosage is reached after a titration of from about 2 to about 10 weeks with the initial dose at about 8 mg increasing to a final dose of about 16 mg to about 24 mg.

14. The regimen of claim 13 wherein said daily dosage is about 24 mg/day, wherein said dosage is reached after a titration of a dose of about 8 mg/day for from about 2 weeks to about 4 weeks, followed by a dose of about 16 mg/day for from about 2 weeks to about 4 weeks, followed by the daily dosage of about 24 mg/day thereafter.

15. The regimen of claim 14 wherein said daily dosage is about 24 mg/day, wherein said dosage is reached after a titration of a dose of about 8 mg/day for about 4 weeks, followed by a dose of about 16 mg/day for about 4 weeks, followed by the daily dosage of about 24 mg/day thereafter.

16. The regimen of claim 13 wherein said daily dosage is about 16 mg/day, wherein said dosage is reached after a titration of a dose of about 8 mg/day for from about 2 weeks to about 4 weeks, followed by the daily dosage of about 16 mg/day thereafter.

17. The regimen of claim 16 wherein said daily dosage is about 16 mg/day, wherein said dosage is reached after a titration of a dose of about 8 mg/day for about 4 weeks, followed by the daily dosage of about 16 mg/day thereafter.

18. Use of a daily dosage of from about 16 mg to about 24 mg of galantamine, wherein said dosage is reached after a titration of from about 2 to about 10 weeks with the initial dose at about 8 mg increasing to a final dose of about 16 mg to about 24 mg for the production of a medicament for treating a galantamine responsive condition.

19. Use of a daily dosage of from about 16 mg to about 24 mg of galantamine, wherein said dosage is reached after a titration of from about 2 to about 10 weeks with the initial dose at about 8 mg increasing to a final dose of about 16 mg to about 24 mg for the production of a medicament for treating Alzheimer's Disease.
